(19) United States
(12) Patent Application Publication
(10) Pub. No.: US 2006/0217443 A1
(43) Pub. Date: Sep. 28, 2006

(54) Method for preventing and/or treating vaginal and vulval infections

(75) Inventors: Lei Huang, Duluth, GA (US); Shu-Ping Yang, Alpharetta, GA (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/091,205

(22) Filed: Mar. 28, 2005

(51) Int. Cl. A61K 31/192

(52) U.S. Cl. 514/568; 514/570

(57) ABSTRACT

The invention provides a method for treating and/or preventing a wide scope of vaginal and vulval infections, such as those caused by bacteria, parasites or yeasts, by administering gallic acid to a subject in need of treatment. A composition containing gallic acid for treating vaginal infections is also disclosed. It has been found that gallic acid is capable of selectively inhibiting the growth of Trichomonas vaginalis, a parasite that causes trichomonas vaginitis; Gardnerella vaginalis, a bacterium that causes bacterial vaginosis; and Candida albicans, a yeast that causes candidiasis and vulvitis; while not inhibiting the growth of Lactobacillus acidophilus, the dominant bacteria in a healthy vaginal ecosystem. Gallic acid is safe and cost-effective, and can be used alone or incorporated into different vaginal health products to treat and/or prevent vaginal infections.

### BACKGROUND OF THE INVENTION

The vaginal ecosystem is a finely balanced environment maintained by a complex interaction among vaginal flora. A variety of bacteria, yeasts and other micro-organisms occur naturally in the vagina's environment. Lactobacillus acidophilus is the dominant bacteria in a healthy vaginal ecosystem, and it maintains an acidic environment of the vagina through the production of lactic acid. Lactic acid and hydrogen peroxide produced by Lactobacilli are toxic to anaerobic bacteria and other pathogenic bacteria in the vagina. The vaginal balance can be upset by external factors such as antibiotics, stress, illness and hormonal changes, and insults that decrease Lactobacilli result in an in overgrowth of pathogenic organisms in the vagina.

More than 75% of women will have at least one vaginal infection in their lives, and 50% of these women will have a recurrence of the infection (http://www.stopgettingsick.com/templates/news_template.cfm/1671). It has been reported that in the United States alone, about 13 million women experience vaginal infections each year.

Bacterial vaginosis, the most common vaginal infection, is caused by an overgrowth of a variety of bacterial species, particularly anaerobes. Gardnerella vaginalis is the main pathogen in bacterial vaginosis.

Bacterial vaginosis generally shows little or no inflammation of the vaginal epithelium and resembles more of an alteration of the bacterial vaginal environment than a real and proper infection of tissues or epithelium. This pathology is currently treated mainly with antibiotics. Antibiotics, however, also kill the useful bacteria, such as Lactobacilli, resulting in a pH increase in the vaginal environment and increasing the risk of recurrence of the bacterial vaginosis or the development of a different vaginal infection, such as a yeast infection. The antibiotics that are usually administered are metronidazole, clindamycin or ampicillin, which are administered orally. This method of use by the systemic route is frequently accompanied by serious side effects. For example, metronidazole exhibits serious side effects, particularly on the blood and on the central nervous system, so much that in certain types of patients it has been necessary to discontinue the treatment, and authorities in the medical field have recommended that women who use metronidazole should not breast feed (Martindale, The Extra Pharmacopoeia, 29th Edition, 1989, page 667). Clindamycin also exhibits serious side effects, particularly on the gastrointestinal tract, with serious forms of diarrhea and pseudo-membranous colitis that can even lead to the death of the patient (Martindale, pages 198-199).

Trichomonas vaginitis, also known as Trichomoniasis or trich, is one of the most common vaginal infections and this infection is considered to be a sexually transmitted disease. In the United States, it is estimated that more than 2 million women are infected with this each year.

Trichomonas vaginitis causes vulvar itching and an odorous vaginal discharge. It is caused by Trichomonas vaginalis, a single-celled protozoan parasite not normally found in the flora of the genitourinary tract. Trichomonas vaginalis is a flagellate protozoa that is pear-shaped and about the size of a white blood cell. These motile cells have four flagellae and a single nucleus. Like bacterial vaginosis, this pathology is generally treated with metronidazole.

The yeast Candida albicans causes the disease known as candidiasis or “thrush”. This is another common vaginal infection, and causes a considerable degree of discomfort. In addition, Candida albicans is also the main pathogen that causes vulvitis, a vulval infection. Candida albicans is present in most humans as a harmless commensal organism, and thousands of the yeast cells can be present in an individual without any ill effect. Problems arise, however, when a person experiences a loss of normal bacterial flora. Candida albicans infection, in severely immune compromised patients, can spread throughout the body and cause deadly systemic infections. Candidiasis is usually treated with fluconazole, but this can have serious side effects and is not recommended for use during pregnancy.

There is therefore a need for a suitable compound or composition that can treat and/or prevent vaginal and vulval infections without killing the useful bacteria and without the side effects of known treatments.

### SUMMARY OF THE INVENTION

In response to the problems discussed above, it has been found that gallic acid is capable of selectively inhibiting and/or killing a wide range of pathogens such as Trichomonas vaginalis, Gardnerella vaginalis and Candida albicans without affecting Lactobacilli growth. This compound is therefore suitable for use as an active ingredient in a method of treating and/or preventing vaginal and vulval infections caused by yeasts, bacteria and protozoa, and in particular, infections such as trichomonas vaginitis, bacterial vaginosis and candidiasis.

According to a first aspect of the invention, a method of treating and/or preventing a vaginal infection using gallic acid is described. The gallic acid may be in the form of a solution, a powder and/or a crystalline structure. The gallic acid may be used alone or in a therapeutic amount in a composition, in the form of a foam, a cream, a gel, a jelly, a moisturizer, a spray, a suppository, a vaginal capsule, a vaginal tablet, a vaginal film, a vaginal sponge, a vaginal ovule or any other vaginal health product. The composition may also be applied to a vaginal insert, tampon, wipe or pad. The composition may further include a suitable diluent, excipient and/or auxiliary.

In general, the gallic acid is present in the composition in an amount of from about 0.05 to about 10 percent (grams/100 milliliter (wt/vol)), more preferably in an amount of from about 0.075 to about 5 percent (wt/vol), even more preferably in an amount of from about 0.1 to about 2 percent (wt/vol), and even more preferably in an amount of from about 0.5 to about 1 percent (wt/vol).

The method comprises the step of administering the composition topically to a subject in need thereof, so as to inhibit the growth of the pathogenic bacteria, yeast or protozoa without inhibiting the growth of Lactobacillus acidophilus.

According to a second aspect of the invention, a composition for treating and/or preventing a vaginal infection is described. The composition comprises a therapeutically effective amount of gallic acid and is substantially as described above.

According to a third aspect of the invention, the use of gallic acid in a method of manufacturing a medicament for treating and/or preventing a vaginal and vulval infection is described.

### BRIEF DESCRIPTION OF THE DRAWINGS

FIG. 1 shows the effect of gallic acid on Trichomonas vaginalis cell counts after 24 hours;

FIG. 2 shows the effect of gallic acid on Trichomonas vaginalis cell counts after 48 hours;

FIG. 3 shows the effect of gallic acid on Candida albicans after 2, 6 and 24 hours; and

FIG. 4 shows the effect of gallic acid on Lactobacillus acidophilus after 2, 6 and 24 hours.

### DETAILED DESCRIPTION OF THE INVENTION

The invention provides a method for treating a vaginal infection by topically administering a therapeutic amount of gallic acid or a pharmaceutically acceptable salt thereof to a subject in need thereof.

Gallic acid or 3,4,5-trihydroxybenzoic acid (C6H2(OH)3CO2H) is a naturally occurring polyphenolic antioxidant found in gallnuts, Sumac, green tea, oak bark, grape seed extract and many other plants. It is a colorless crystalline organic acid found both in its free state and as part of the tannin molecule. Gallic acid has hydroxyl groups and a carboxylic acid group in the same molecule, and thus two molecules can react with one another to form an ester, digallic acid. Gallic acid is obtained by the hydrolysis of tannic acid with sulfuric acid. Salts of gallic acid include any physiologically acceptable salt available to one of skill in art. Examples include sodium, calcium or potassium salts of gallic acid.

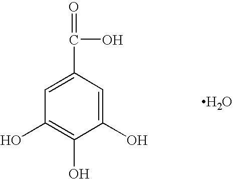

### Chemical Structure of Gallic Acid Monohydrate

It has previously been shown that gallic acid does not affect the growth of Lactobacillus acidophilus, the beneficial flora in the vagina.

The applicant has now surprisingly found that gallic acid has the effect of selectively inhibiting the parasite Trichomonas vaginalis, the bacterium Gardnerella vaginalis and the yeast Candida albicans while not inhibiting Lactobacillus acidophilus, and it is therefore a suitable compound for treating and/or preventing vaginal infections, and in particular, trichomonas vaginitis, bacterial vaginosis, candidiasis and symptomatic yeast vaginitis.

The gallic acid or pharmaceutically acceptable salt thereof can be used in the form of a solution, powder and/or crystalline structure, either alone or as part of a composition. It is typically administered topically as part of a composition which is in the form of a foam, cream, gel, jelly, moisturizer, spray, suppository, vaginal capsule, vaginal tablet, vaginal film, vaginal sponge, vaginal ovule or any other vaginal health product. The composition optionally also includes suitable diluents, excipients and/or auxiliaries, which are well known in the art.

The composition can be applied to a vaginal insert, tampon, wipe or pad or can be used on its own.

In general, the gallic acid is present in the composition in an amount of from about 0.05 to about 10 percent (grams/100 milliliter (wt/vol)), more preferably in an amount of from about 0.075 to about 5 percent (wt/vol), even more preferably in an amount of from about 0.1 to about 2 percent (wt/vol), and even more preferably in an amount of from about 0.5 to about 1 percent (wt/vol).

The present invention is further described by the following examples. Such examples, however, are not to be construed as limiting in any way either the spirit or scope of the invention.

### EXAMPLES

Microorganisms and Culture Media:

A sample of Trichomonas vaginalis, the parasite found in trichomonas vaginitis, was obtained from the American Type Culture Collection (ATCC), catalog number 30001. The culture medium was LYI-S-2 medium (ATCC medium 2154).

A sample of Lactobacillus acidophilus, a desirable bacterium in the vaginal ecosystem, was also obtained from the American Type Culture Collection (ATCC), catalog number 4354, and was cultured in ATCC medium 416.

A sample of Gardnerella vaginalis, the pathogenic bacterium found in bacterial vaginosis, was obtained from the American Type Culture Collection (ATCC), catalog number 14018. The culture medium was ATCC medium 70 and Casman's medium (BD 229010) with 5 percent rabbit blood.

A sample of Candida albicans, the yeast found in candidiasis, was also obtained from the American Type Culture Collection (ATCC), catalog number 10231, and was cultured in YM agar (Difco 0712) and YM broth (Difco 0711).

### Example 1

### Effect of 1.1 Percent Gallic Acid on the Growth of Trichomonas vaginalis

A sterile LYI-S-2 medium was prepared according to the manufacturer's instructions, and the pH of this medium was adjusted to pH 6.0 using 1 N HCl. Gallic acid monohydrate (SIGMA-398225 from Signa Aldrich, USA) was dissolved in the LYI-S-2 medium. The gallic acid monohydrate was found to be only partially water soluble, and the highest concentration of gallic acid that could be obtained in water was 1.1 percent (g/100 milliliter). 0.9 milliliter of the gallic acid/LYI-S-2 solution or culture medium only (as control) was added into different culture tubes.

0.1 milliliter of Trichomonas vaginalis culture suspension, at a concentration of 1×10⁶/milliliter, was added to each of the culture tubes, which were then incubated at 35 degrees Celsius on a 15 degree horizontal slant.

The viable Trichomonas vaginalis cells in each tube were counted under a microscope after 24 hours.

This procedure was repeated twice.

The results in Table 1 show that gallic acid at a concentration of 1.1 percent completely reduced the Trichomonas vaginalis cell count after 24 hours and no live Trichomonas vaginalis cells were observed in the treatment group. In contrast, between 0.9 and 1 million live Trichomonas vaginalis cells were counted in the control group.

TABLE 1
Number of Trichomonas cells with
or without gallic acid treatment

| | Repeat 1 | Repeat 2 |
|---|---|---|
| Culture medium only | 900 000 | 1 000 000 |
| (control) | | |
| Gallic acid (1.1 percent) | 0 | 0 |

### Example 2

### Effect of Various Concentrations of Gallic Acid on the Growth of Trichomonas vaginalis

Essentially the same procedure was performed as described in Example 1, but this time differing concentrations of gallic acid were tested on samples of Trichomonas vaginalis. The gallic acid concentrations that were tested were: 0.11 percent, 0.66 percent and 1.1 percent. More samples in each group were included (n=4), and the effects of the gallic acid over a longer time period were also observed, i.e. after 24 and 48 hours.

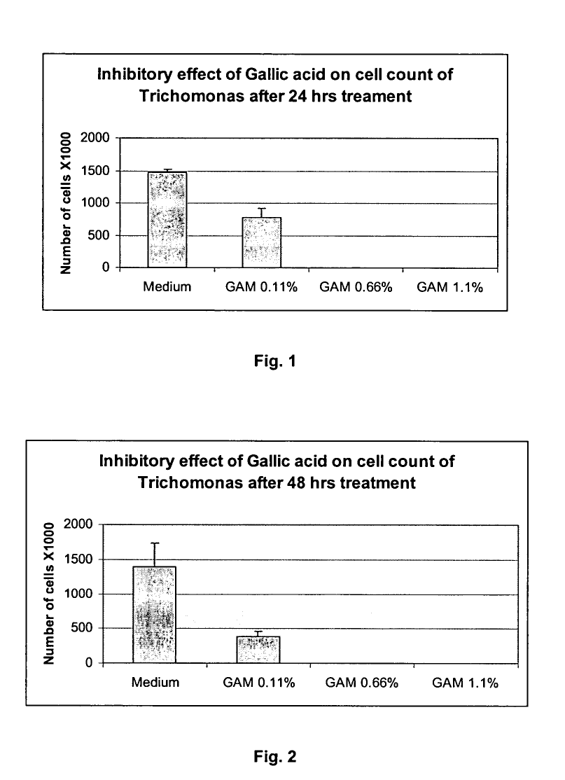

As shown in FIGS. 1 and 2, gallic acid at a concentration of 0.11 percent showed a 50 to 70 percent inhibitory effect on Trichomonas vaginalis cell counts after 24 and 48 hours, respectively, compared to the control group (medium only). No live Trichomonas vaginalis cells were observed in the samples that were treated with gallic acid concentrations of 0.66 or 1.1 percent, either after 24 or 48 hours.

### Example 3

### Effect of Gallic Acid on the Growth of Candida albicans and Lactobacillus acidophilus—Zone-of-Inhibition Test

A microorganism culture of 10⁵ cfu (colony forming units)/milliliter in a 1× phosphate buffered saline (PBS) solution (diluted from 10× PBS LIQUID CONCENTRATE from VWR Cat. No. EM-6507] was prepared for each of Candida albicans and Lactobacillus acidophilus. One milliliter of each solution was plated on proper agar plates, depending on which microorganism was being tested. The agar plates were incubated at 35 degrees Celsius for four hours. Three 4 millimeter diameter wells were then punched into each agar plate. A test sample of 10 mg/ml gallic acid in sterilized 2-N-morpholino ethane sulfonic (MES, pH=4.7) buffer (0.1 M 2-[morpholino]-ethanesulfonic acid, 0.9 percent NaCl, pH 4.7, prepared from BupH™ MES Buffer Saline Pack from Cat. No. 28390, Pierce Biotechnology, Inc., Rockford, Ill.) was added to one well of each plate. Into each of the other two wells were added MES buffer and 0.5 percent Benzyl Quats (diluted from BARDAC® 205M, from Lonza Inc., Fair Lawn, N.J.) as negative and positive controls, respectively. The plates were incubated overnight at 35 degrees Celsius. The presence of a zone of microorganism inhibition was measured the following day for Candida albicans and Lactobacillus acidophilus activity, respectively.

As shown in Table 2, gallic acid at a concentration of 10 mg/ml selectively inhibited Candida albicans, while it did not affect the growth of Lactobacillus acidophilus. The positive control, 0.5 percent Benzyl Quats, inhibited the growth of both microorganisms, while MES buffer itself had no effect on either of the two microorganisms.

TABLE 2
Effect of gallic acid on Candida albicans and Lactobacillus acidophilus
with zone of inhibition test, n = 2.

| Tested | | |
|---|---|---|
| Compounds/Polymers | Candida albicans | Lactobacillus acidophilus |
| 10 milligram/ml gallic acid | 5 millimeter | 0 millimeter |
| 0.5 percent Benzyl Quats | 5 millimeter | 7 millimeter |
| MES buffer | 0 millimeter | 0 millimeter |

### Example 4

### Effect of Gallic Acid on the Growth of Candida albicans and Lactobacillus acidophilus—Inhibition Tests in Solution by Measuring Optical Density

Test compounds of Candida albicans and Lactobacillus acidophilus were dissolved in culture media to form a suspension. Control and gallic acid solutions (0.9 milliliters) were filtered and added into separate culture tubes, and to these were added 0.1 milliliter of either the Candida albicans or Lactobacillus acidophilus suspension at a concentration of around 10⁶ cfu/milliliter. The culture tubes were then incubated overnight at 37 degrees Celsius, whereafter the optical density was measured at 2, 4, 6 and 24 hours at 590 nanometers, by pippeting 100 microliters of the control or sample solutions into 96-well microplates, and then using a Molecular Devices of Sunnyvale, Calif. ThermoMax Microplate Reader to obtain the optical density readings at 590 nm wavelengths.

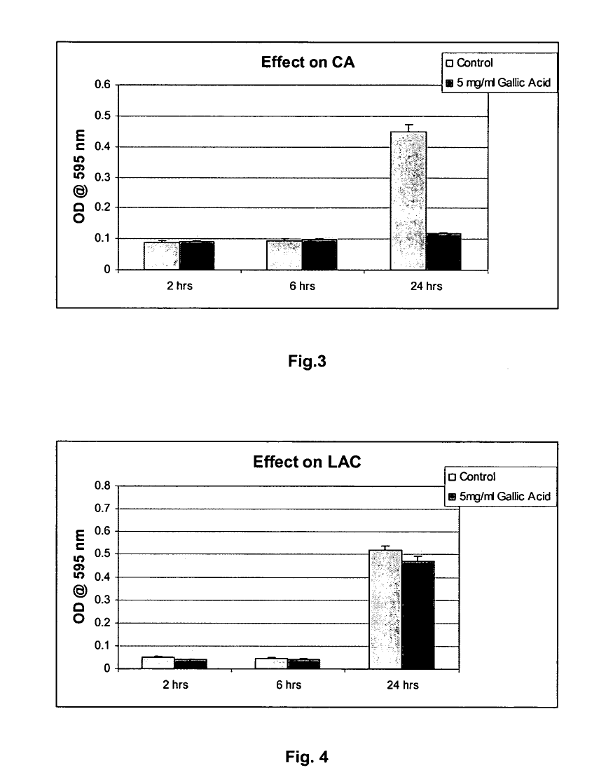

Gallic acid at a concentration of 5 milligram/milliliter was shown to significantly inhibit the growth of Candida albicans after 24 hours of treatment (FIG. 3).

In contrast to the profound inhibition on the growth of Candida albicans, gallic acid at the same concentration did not show any significant inhibition on the growth of Lactobacillus acidophilus after 24 hours (FIG. 4).

These optical density results were consistent with the zone-of-inhibition results of Example 3.

### Example 5

### Effect of Gallic Acid on the Growth of Gardnerella vaginalis—Inhibition Test in Solution by Plate Count

A test compound of Gardnerella vaginalis was prepared in culture media to form a suspension. Control and gallic acid solutions (0.9 milliliters; 7.5 milligram gallic acid/milliliter) were filtered and added into separate culture tubes, and to these were added 0.1 milliliter of the Gardnerella vaginalis suspension at a concentration of around 10⁶ cfu/milliliter. The culture tubes were incubated at 37 degrees Celsius for 24 hours.

The samples in the culture tubes were then diluted at 1, 10 and 100 times, and 100 microliters of each dilution was plated onto agar plates with WASP (Whitely Automatic Spiral Plate) spiral plating equipment from Don Whitely Scientific Limited, USA. The plates were incubated overnight at 35 degrees Celsius, and the colonies were counted on each plate by either ProtoCol® from Synbiosis, Frederick, Md., USA Whitely Scientific Limited, USA or by hand count.

After 24 hours of treatment, gallic acid showed significant inhibition (over 99.98 percent) on the growth of Gardnerella vaginalis compared to a control group (Table 3).

TABLE 3
Effect of gallic acid on Gardnerella vaginalis
after 24 hours treatment, n = 4

| | Negative control | 7.5 mg/ml gallic acid |
|---|---|---|
| Average | 1.81E+08 | *3.43E+04 |
| STDEV | 6.36E+06 | 2.12E+03 |

*represents p < 0.05 compared to negative control group

The results of the above examples clearly demonstrate that different concentrations of gallic acid are able to effectively inhibit Trichomonas vaginilis, Gardnerella vaginalis and Candida albicans. As gallic acid has also been shown not to inhibit the growth of Lactobacillus acidophilus, and as it is a naturally occurring, safe compound which is also cost-effective, it is ideally suited to be formulated into vaginal health products, such as tampons, pads, wipes, vaginal moisturizers, sprays, gels and so forth for preventing and/or treating vaginal infections such as trichomonas vaginitis, bacterial vaginosis and candidiasis.

While the invention has been described in detail with respect to specific embodiments thereof, it will be appreciated by those skilled in the art that various alterations, modifications and other changes may be made to the invention without departing from the spirit and scope of the present invention. It is therefore intended that the claims cover or encompass all such modifications, alterations and/or changes.

We claim:

1. A method of treatment of a vaginal or vulval infection which comprises administering topically to a subject in need of treatment a therapeutically effective amount of gallic acid or a pharmaceutically acceptable salt thereof.

2. The method of claim 1, wherein the infection is selected from the group consisting of trichomonas vaginitis, bacterial vaginosis, candidiasis and vulvitis.

3. The method of claim 1, wherein the gallic acid is part of a composition selected from the group consisting of a foam, a cream, a gel, a jelly, a moisturizer, a spray, a suppository, a vaginal capsule, a vaginal tablet, a vaginal film, a vaginal sponge, a vaginal ovule, a vaginal insert, a tampon, a wipe and a pad.

4. The method of claim 1, wherein the gallic acid is in a form selected from the group consisting of a solution, a powder and a crystal structure.

5. The method of claim 1, wherein the treatment inhibits the growth of a pathogen selected from the group consisting of Trichomonas vaginalis, Garderella vaginalis and Candida albicans and does not inhibit the growth of Lactobacillus acidophilus.

6. The method of claim 1, wherein the gallic acid is present in the composition in an amount of from about 0.05 to about 10 percent (wt/vol).

7. A method of preventing a vaginal or vulval infection which comprises administering topically to a subject in need of treatment a therapeutically effective amount of gallic acid or a pharmaceutically acceptable salt thereof.

8. The method of claim 7, wherein infection is selected from the group consisting of trichomonas vaginitis, bacterial vaginosis, candidiasis and vulvitis.

9. The method of claim 7, wherein the gallic acid is part of a composition selected from the group consisting of a foam, a cream, a gel, a jelly, a moisturizer, a spray, a suppository, a vaginal capsule, a vaginal tablet, a vaginal film, a vaginal sponge, a vaginal ovule, a vaginal insert, a tampon, a wipe and a pad.

10. The method of claim 7, wherein the gallic acid is in a form selected from the group consisting of a solution, a powder and a crystal structure.

11. The method of claim 7, wherein the treatment inhibits the growth of a pathogen selected from the group consisting of Trichomonas vaginalis, Garderella vaginalis and Candida albicans and does not inhibit the growth of Lactobacillus acidophilus.

12. The method of claim 7, wherein the gallic acid is present in the composition in an amount of from about 0.05 to about 10 percent (wt/vol).

13. The use of gallic acid or a pharmaceutically acceptable salt thereof in a method of making a medicament for use in a method of preventing or treating a vaginal or vulval infection comprising administering topically a therapeutically effective amount of the gallic acid to a patient in need thereof.

14. The use of claim 13, wherein the infection is selected from the group consisting of trichomonas vaginitis, bacterial vaginosis, candidiasis and vulvitis.

15. The use of gallic acid according to claim 13, wherein the gallic acid is in a form selected from the group consisting of a solution, a powder and a crystal structure.

16. The use of gallic acid according to claim 13, wherein the medicament is in the form of a foam, a cream, a gel, a jelly, a moisturizer, a spray, a suppository, a vaginal capsule, a vaginal tablet, a vaginal film, a vaginal sponge, a vaginal ovule, a vaginal insert, a tampon, a wipe and a pad.

17. The use of gallic acid according to claim 13, wherein the medicament is applied to a tampon, pad, wipe or vaginal insert.

18. The use of gallic acid according to claim 13, wherein the gallic acid is present in the medicament in an amount of from about 0.05 to about 10 percent (wt/vol).

19. The use of gallic acid according to claim 13, wherein the medicament inhibits the growth of Trichomonas vaginalis and does not inhibit the growth of Lactobacillus acidophilus.

20. A composition for preventing or treating a vaginal or vulval infection, which comprises a therapeutically effective amount of gallic acid or a pharmaceutically acceptable salt thereof.
